(19) United States
(12) Patent Application Publication
(10) Pub. No.: US 2024/0261286 A1
(43) Pub. Date: Aug. 8, 2024

(54) BIOFACTORS FOR THE TREATMENT AND PROPHYLAXIS OF DEMENTIA

(71) Applicant: WÖRWAG Pharma GmbH & Co.KG, Böblingen (DE)

(72) Inventors: Fritz WÖRWAG, Stuttgart (DE); Marcus WÖRWAG, Stuttgart (DE)

(21) Appl. No.: 18/030,740

(22) PCT Filed: Oct. 7, 2021
(86) PCT No.: PCT/EP2021/077684

§ 371 Date: Oct. 16, 2023

(30) Foreign Application Priority Data
Oct. 7, 2020 (EP) 20200644.1

(51) Int. Cl. A61K 31/513; A61K 31/519; A61K 31/593; A61K 31/675; A61K 31/714; A61P 25/28

(52) U.S. Cl. CPC A61K 31/513; A61K 31/519; A61K 31/593; A61K 31/675; A61K 31/714; A61P 25/28

(57) ABSTRACT

The invention relates to an agent for the treatment and prophylaxis of dementia (diseases) and mitochondrial dysfunction, in particular a drug or a dietary supplement and the use thereof, which contains a combination of biofactors, at least comprising magnesium orotate and folic acid or folates.

The invention relates to an agent for the treatment and prophylaxis of dementia (diseases) and mitochondrial dysfunction, in particular a drug or a dietary supplement and the use thereof, which contains a combination of biofactors.

Dementia is classified as F00-F03 in the ICD-10 (International Statistical Classification of Diseases and Related Health Problems, 10th Revision (2019)) by the World Health Organization (WHO) in Chapter V, “Mental and behavioural disorders”, and is a syndrome due to disease of the brain, usually of a chronic or progressive nature, in which there is disturbance of multiple higher cortical functions, including memory, thinking, orientation, comprehension, calculation, learning capacity, language, and judgement. Consciousness is not clouded. The impairments of cognitive function are commonly accompanied, and occasionally preceded, by deterioration in emotional control, social behaviour, or motivation. This syndrome occurs in Alzheimer's disease, in cerebrovascular disease, and in other conditions primarily (directly) or secondarily (indirectly) affecting the brain (see also “Dementia: A Public Health Priority” (2012) (https://www.alz.co.uk/WHO-dementia-report)).

Alzheimer's dementia (AD) is the most common form of dementia and today affects more than 60% of people with dementia worldwide. The main pathological characteristic of AD is the formation of senile or amyloid plaques, consisting of the AB peptide, and neurofibrillary aggregates accompanied by synaptic and neural degeneration and gliosis, with hyperphosphorylated microtubules being associated with the tau protein (MAPT). The AB peptide results from the activity of at least two different proteases from a precursor protein, the “amyloid precursor protein” (APP), which is located in the cell wall of neurons. During the proteolytic breakdown of APP and due to subsequent modification, AB fragments of different lengths and types result. The deposition of AB in the form of plaques is still considered to be at least one trigger of the disease symptoms. In vascular dementia, circulatory disorders in the brain result in neurons dying. These disorders may for example be the result of multiple minor strokes (e.g. due to vascular occlusion), which may likewise occur in a delayed manner (known as “multi-infarct dementia”).

Until now, at best, the symptoms of dementia such as AD, which result from neurons dying and determine the degree of dementia, can be treated, but not the cause.

Today, no approved medications are known which can cure the disease processes in (primary) dementia caused by a disease of the brain.

The disease progression or even the onset of dementia can be delayed or positively influenced, however. In addition to reducing the individual risk (e.g. preventing excess weight (obesity), lack of exercise, smoking, alcohol, negative stress, etc.), biofactors, such as vitamins and the like, can make an effective contribution, with long-term and continuous treatment or prophylaxis being intended to be pursued. This is the focus of the applicant's research.

AD is a multifactorial disease in which the role of metabolic and inflammatory comorbidities appears to be becoming increasingly significant. Just ˜2% of cases of AD are caused by somatic mutations in the amyloid precursor protein (APP), presenilin 1 (PSEN1) and presenilin 2 (PSEN2) (familial AD, fAD), while 98% of cases of AD have a sporadic (sporadic AD, sAD), as yet unknown, but complex and multifactorial aetiology [1-3]. Genome-wide association studies (GWAS) have previously linked alleles of apolipoprotein E, more specifically ApoE E4, to an increased risk of developing both mild cognitive impairment and AD [4, 5]. Other genetic factors that have been identified on the basis of GWAS and have been linked to the occurrence of sAD are e.g. represented by apolipoprotein J (APOJ), phosphatidylinositol binding clathrin assembly protein (PICALM), triggering receptor expressed on myeloid cells 2 (TREM2), cluster of differentiation 33 (CD33), and complement receptor 1 (CR1) [4-7]. Recently, metabolic comorbidities of AD, caused by malnutrition, in particular in Western countries, have been attributed an increasingly significant role [8, 9]. One of the comorbidities of sAD that has been identified so far is diabetes (type 1 or type 2), a pathological condition widespread in population groups in the Western world, with a current estimate of 176 million people worldwide being affected by type 1 diabetes (˜5-10% of cases), type 2 diabetes (˜90% of cases) or other forms of diabetes, e.g. gestational diabetes [9, 10]. Type 2 diabetes, which is responsible for the majority of diabetes cases worldwide, occurs more frequently in older people and is linked to energy-rich, low-quality food as part of a sedentary lifestyle—circumstances that are prevalent in population groups in Western industrialised countries. The diet in these countries often includes cheap, highly processed foods, which contain a significant proportion of saturated fatty acids, cholesterol and simple carbohydrates and at the same time lack essential minerals, trace elements and vitamins [11-14]; for example, in Western population groups and in particular among older people, there is often in particular a chronic deficiency of magnesium, zinc and vitamin B, in particular thiamine, folic acid or prodrugs such as benfotiamine and vitamin D, with a deficiency extending through all stages, from asymptomatic stages, which can only be determined under laboratory conditions, through to clinically manifest deficiencies with or without specific symptoms. It is noteworthy that ˜80% of AD patients develop a glucose intolerance or diabetes in the course of dementia [15, 16], which points towards mutual interaction [17]. Mitochondrial dysfunction can be considered to be associated therewith, which may be a cause of or contributory factor to dementia.

In addition, Western diets in combination with a lack of physical activity often result in obesity [14], and in particular an increased concentration of visceral fat causes metainflammation [18] (known as “cold inflammation”), which results in the release of pro-inflammatory cytokines IL1β and TNF-α from infiltrating macrophages and hepatocytes and thus systemic metabolic deterioration takes place [18, 19]. Both obesity and diabetes exacerbate or “fuel” the pathology of AD [9, 11, 20].

In view of the fact that, in a population that is ageing overall, increasing numbers of patients are suffering from metabolic disorders, including diabetes and obesity (which are primarily caused by malnutrition in isolation or in combination with a lack of activity), which are a major risk factor for the development of dementia, in particular AD, a deficiency or reduced intake of biofactors, in particular magnesium, zinc and vitamin B, in particular thiamine, folic acid or prodrugs such as benfotiamine and vitamin D, are a high risk for older people. This is associated with altered metabolic process in older people (e.g. muscular atrophy, etc.).

Biofactors for the treatment and prophylaxis of dementia are described in the prior art. For example, even a vitamin deficiency, in particular a deficiency of vitamin B12, can cause (secondary) dementia (WHO ICD10 (2019), F02.8).

As explained above, the applicant is focusing in particular on the following compounds or substances, and specifically magnesium or zinc orotate, vitamin D, in particular vitamin D3 (cholecalciferol) and vitamin B, in particular thiamine, folic acid, vitamin B6 and B12 (cobalamin) and benfotiamine.

In particular, orotates have proven to be promising Zn and Mg vehicles, since they can easily cross the blood-brain barrier. Orotates are also advantageous since e.g. in particular both magnesium orotate and zinc orotate can be easily conducted through or permeate the cell membrane, and can also preferably be absorbed by mitochondria and the nucleus [21]. Resulting stimulation of the energy supply, therefore e.g. the ATP production in mitochondria, is advantageous since dementia, in particular AD, can have mitochondrial dysfunction as a cause or contributory factor (supra).

Furthermore, combination preparations of the above biofactors for the treatment and prophylaxis of dementia are described in the prior art.

US20150132273A1 thus describes magnesium threonate in combination with biofactors for the treatment of cognitive impairment. Threonate has, however, proven to be an ineffective vehicle for magnesium or zinc, since threonate itself is rapidly broken down via several metabolic pathways.

The use of magnesium salts, but not magnesium orotate, in combination with biofactors for the treatment of dementia is described in WO 2017/179644 A1.

WO 2017/059895 A1 discloses, in example 1, a preparation composed of L-5 methyltetrahydrofolate (100-800 μg folic acid equivalent), vitamin B3 (4-40 mg), vitamin B2 (0.4-5 mg), methylcobalamin (0.5-10 μg), vitamin B6 (0.4-5 mg), trimethylglycine (100-2000 mg), zinc bisglycinate (5-50 mg) and N-acetyl cysteine (100-2000 mg) for the treatment of dementia.

Mischoulon et al [22] discloses folate for the treatment of dementia, including senile Alzheimer's dementia, and for the prophylaxis thereof. Administration of 15 mg per day to patients is described.

Proceeding from this prior art, the problem addressed by the present invention is therefore to provide particularly suitable and effective combinations of biofactors for the treatment and prophylaxis of dementia, in particular for long-term therapy or long-term prophylaxis.

For this purpose, the applicant has performed comprehensive tests on preclinical AD models, which demonstrate the suitability of biofactor combinations for the prophylaxis and treatment of dementia, in particular AD.

The immortalised neuroblastoma cells, and specifically human SY-SY5Y-APP695 cells, overexpress the neural human APP gene, resulting in the increased formation of B amyloid.

The nematode C. elegans (CL2006) expresses human amyloid B (1-42) under the control of a muscle-specific promoter, such that progressive paralysis of the worm takes place. The lifespan is reduced and the worm exhibits characteristic deposits of B amyloid.

Alzheimer's rats TgF344-AD were tested after 6-7 and 15-16 months for activity of oxidative phosphorylation of the mitochondria compared with wild types.

The results are explained and evidenced in the examples and the drawings.

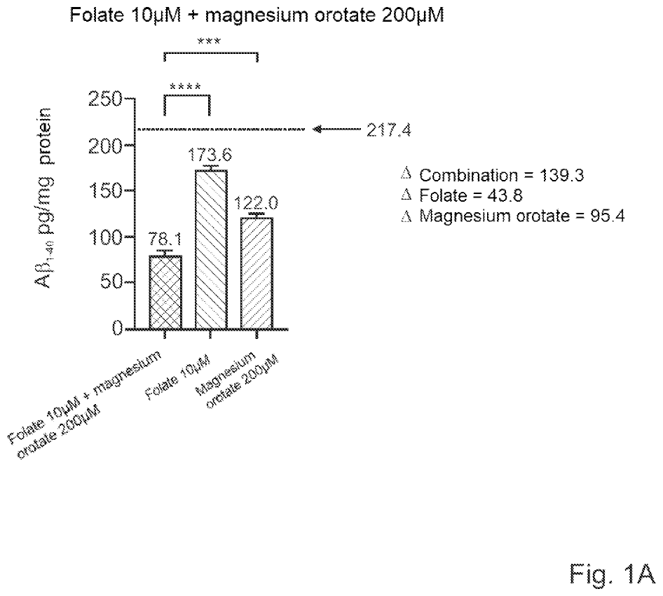

Surprisingly, for a composition of magnesium orotate and folic acid in immortalised neuroblastoma cells, and specifically human SY-SY5Y-APP695 cells, over-additive inhibition of B amyloid could be achieved (FIG. 1A).

This result is in particular also seen in the suitability of this combination for normalising the ATP production in the mitochondria and counteracting causative mitochondrial dysfunction.

Therefore, the invention relates to an agent, in particular a drug or a dietary supplement or a balanced diet, comprising or consisting of magnesium orotate and folic acid for use in the prophylaxis and treatment of dementia (in the following: agent according to the invention).

In another embodiment, the invention therefore relates to an agent, in particular a drug or a dietary supplement or a balanced diet, comprising or consisting of magnesium orotate and folic acid for use in the prophylaxis and treatment of mitochondrial dysfunction, in particular those selected from the group of heart failure, non-alcoholic hepatitis, chronic fatigue, or fibromyalgia (in the following: agent according to the invention).

The above-mentioned tests demonstrate that, in addition to magnesium orotate and folic acid, in particular a “cocktail” of magnesium or zinc orotate, vitamin D, in particular vitamin D3 (cholecalciferol) and vitamin B, in particular thiamine, folic acid, vitamin B6 and B12 (cobalamin) and benfotiamine at least additively support the effect.

Therefore, other combinations are also included according to the invention, with at least two or three or four or five or six substances or all the substances being selected from the group of magnesium orotate, zinc orotate, vitamin D3, vitamin B6, folic acid, vitamin B12 and benfotiamine.

Magnesium orotate is salt of orotic acid (or 6-carboxyuracil) having the formula

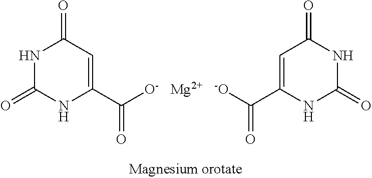

and is commercially available, e.g. from Worwag Pharma GmbH (Böblingen, Germany).

Zinc orotate is salt of orotic acid (or 6-carboxyuracil) having the formula

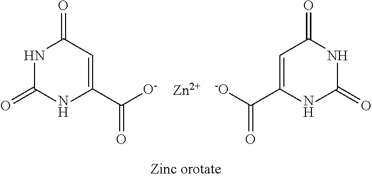

and is commercially available, e.g. from Wörwag Pharma GmbH (Böblingen, Germany).

Folic acid (or pteroylmonoglutamic acid) is vitamin B9

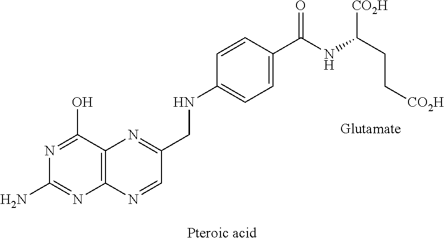 [SMILES: Nc1nc(O)c2nc(CNc3ccc(C(=O)N[C@@H](CCC(=O)O)C(=O)O)cc3)cnc2n1]

or the tautomers (2S)-2-[(4-[(2-amino-4-oxo-1H-pteridin-6-yl)methylamino]benzoyl)amino]pentanedioic acid (lactam) or (2S)-N-(4-[([2-amino-4-hydroxypteridin-6-yl]methyl)amino]benzoyl)glutamic acid (lactim) and can be present as folates, with there being 2-7 glutamyl residues. In addition, folates can be hydrogenated on the pteridine, such as 5, 6, 7, 8 tetrahydrofolate, or can be substituted (e.g. methylfolate, such as 5-methyltetrahydrofolate, inter alia).

According to the invention, such folates (or folic-acid equivalents) are covered by the term folic acid. Also according to the invention, tautomers and salts of folic acid are also covered. The synthetically prepared folic acid is preferred according to the invention.

Benfotiamine is a lipophilic prodrug of thiamine (vitamin B1) and is thus a provitamin and is e.g. distributed by the applicant as milgamma protekt®, and specifically for the treatment of neuropathies and cardiovascular diseases caused by vitamin B1 deficiency.

In another preferred embodiment of the agent according to the invention, the molar ratio of magnesium orotate to folic acid is preferably 2-25:1, in particular 20:1.

Vitamins B and D relate to the commercially available vitamins D2 and D3 and B1 to B12.

Said substances can be used in their physiologically active forms, and can contain salts, cofactors and hydrates or stereoisomers, tautomers (e.g. R, S, Z, E), etc.

In the context of this invention, dementia (disease(s)) are understood (not exhaustively) to be those such as Alzheimer's dementia or Alzheimer's disease, dementia in Alzheimer's disease with early onset (type 2), dementia in Alzheimer's disease with late onset (type 1), dementia in Alzheimer's disease, atypical or mixed type, vascular dementia, and Lewy body dementia, with all these forms being covered according to the invention. These forms of dementia are described in the “International Statistical Classification of Diseases and Related Health Problems”, 10th Revision (2019) by the World Health Organization (WHO) in Chapter V, “Mental and behavioural disorders” as F00-03 (supra).

In the context of this invention, the term “mitochondrial dysfunction” is understood to mean that there is an imbalance between the formation of energy (ATP and NADH) and the formation of oxygen radicals or ROS (reactive oxygen species) in a cell or in the mitochondria therein, such that this results in a lack of energy and increased oxidative stress and secondary diseases, in particular chronic diseases, are or can be caused, such as dementia, in particular AD, heart failure, non-alcoholic hepatitis, chronic fatigue, chronic fatigue syndrome (CFS), or fibromyalgia (syndrome). Evidence is provided for a causal relationship between mitochondrial dysfunction and AD on the basis of the literature [23, 24], and also for chronic fatigue, CFS, or fibromyalgia [25].

All of said indications are described in Pschyrembel, 267th edition 2017, De Gruyter (Berlin).

The effective agent containing the combination of magnesium orotate and folic acid according to the invention can now advantageously be used for the treatment and prophylaxis of an ill patient or individual, animal, mammal or preferably human, and specifically for the treatment and prophylaxis of dementia (diseases), mitochondrial dysfunction, heart failure, non-alcoholic hepatitis, chronic fatigue, chronic fatigue syndrome (CFS), or fibromyalgia (syndrome).

The agents can be administered in any desired quantities and dosages.

The galenic formulation of the agent according to the invention can be selected from the group consisting of: drops, juice, syrup, tablets, sugar-coated tablets, capsules, sustained-release formulations, infusions, ointments, emulsions, or powder. The formulation may of course contain pharmaceutically standard excipients.

In another embodiment, the invention relates to a drug containing an agent according to the invention for use or application for the treatment and prophylaxis of dementia (diseases), mitochondrial dysfunction, heart failure, non-alcoholic hepatitis, chronic fatigue, chronic fatigue syndrome (CFS), or fibromyalgia (syndrome).

Another preferred embodiment relates to a dietary supplement containing the agent according to the invention, in particular in the form of a dietary composition or balanced diet for the treatment and prophylaxis of dementia (diseases), mitochondrial dysfunction, heart failure, non-alcoholic hepatitis, chronic fatigue, chronic fatigue syndrome (CFS), or fibromyalgia (syndrome). Suitable food and foodstuffs according to the invention, including water, are e.g. those as defined (not exhaustively) in Regulation (EC) No 178/2002 of 28 Jan. 2002, and those such as baked goods and beverages and baby food preparations. A suitable physiologically compatible carrier can be added to the dietary supplement according to the invention.

The pharmaceutical preparations according to the invention may be prepared in the form of dosage units. This means that the preparations are in the form of individual parts, preferably capsules and vials, the active-substance content of which corresponds to a fraction or multiple of an individual dose.

The dosage units may e.g. contain 1, 2, 3 or 4 individual doses or ½, ⅓ or ¼ of an individual dose. An individual dose preferably contains the quantity of the agent according to the invention that is administered in an application and usually corresponds to a whole daily dose, half of a daily dose, a third of a daily dose or a quarter of a daily dose. Preferably, a dose is administered three times per day, preferably in the form of a tablet or drops, in particular in the morning, at lunchtime and in the evening, optionally at mealtimes.

Non-toxic, inert, pharmaceutically suitable carrier substances are understood to be solid, semi-solid or liquid dilution agents, fillers and formulation excipients of any type, such as a) fillers and extenders, e.g. starches, lactose, sucrose, glucose, mannitol, dextrins, maltodextrin and silica, highly dispersed silicon dioxide, b) binders, e.g. carboxymethyl cellulose, cellulose powder, microcrystalline cellulose, alginates, gelatine, polyvinylpyrrolidone, c) humectants, e.g. glycerol, d) disintegrants, e.g. agar, calcium carbonate and sodium carbonate, e) dissolution retardants, e.g. paraffin, and f) resorption accelerators, e.g. quaternary ammonium compounds, g) wetting agents, e.g. cetyl alcohol, glycerol monostearate, h) adsorbents, e.g. kaolin and bentonite, and i) lubricants, e.g. talc, calcium and magnesium stearate, and solid polyethylene glycols or mixtures of the substances set out in a) to i).

The tablets, sugar-coated tablets, pills and granulates can be provided with the standard coatings and shells, optionally containing opacifiers, e.g. those such as (not exhaustively) hypromellose, microcrystalline cellulose, stearic acid, titanium dioxide, and can likewise be composed such that they only or preferably release the active substance(s) in a certain part of the intestinal tract where required in a delayed manner, with it e.g. being possible to use polymer substances and waxes as embedding compounds.

Therefore, the invention likewise relates to a pharmaceutical preparation containing an agent according to the invention together with excipients and additives.

### EXAMPLES AND DRAWINGS

The following examples and drawings are used solely to explain the invention, without limiting the invention to these examples.

### Example 1

### Cell Model:

### Cell Cultivation:

The human neuroblastoma SH-SY5Y cells used were transfected in a stable manner with DNA of the human wild type APP695 (APP) (Grewal R, Reutzel M, Dilberger B et al. (2020) Purified oleocanthal and ligstroside protect against mitochondrial dysfunction in models of early Alzheimer's disease and brain ageing. Exp Neurol 328: 113248, Stockburger C, Gold VAM, Pallas T et al. (2014) A cell model for the initial phase of sporadic Alzheimer's disease. J Alzheimers Dis 42: 395-411).

SH-SY5Y-APP cells were cultivated at 37° C. in an atmosphere of 5% CO2 in a culture medium (DMEM), supplemented with 10% (v/v) heat-inactivated foetal calf serum, 60 μg/ml streptomycin, 60 units/ml penicillin, 0.3 mg/ml hygromycin, non-essential MEM amino acids, and 1 mM sodium pyruvate 1%. The cells were subcultured every 3 days and were used when they reached 70-80% confluency.

The cells were incubated for 24 hours with 200 μM magnesium orotate, 10 μM folic acid and the combination thereof. An incubation with DMEM was used as a control.

### Aβ 1-40 Measurement:

After 24 hours of incubation, the medium was collected in the cell-culture medium bottles and the cells were rinsed with cold PBS. The suspension was then centrifuged for 5 minutes at 220 g. The supernatant was discarded and the cell pellets were resuspended in 1.5 ml PBS and protease inhibitor. The solution was then centrifuged for 5 minutes at 112 g and the supernatant was discarded. The cell pellet was collected and 600 μl cell extraction buffer (Thermo Fisher Scientific, Waltham, MA, USA) was added and was incubated for 30 minutes. The suspension was then centrifuged for 10 minutes at 13,000 g. The supernatant was transferred into a new vial and stored at −80° C. The supernatants were defrosted on ice and were pipetted into a 384-well plate (Greiner Bio-One, Kremsmunster, Austria). In order to establish the amyloid beta concentration, an HTRF amyloid beta 1-40 kit (Cisbio, Codolet, France) was used. The samples were used in accordance with the manufacturer's instructions. The optical density was then measured at 665 and 622 nm. The amyloid beta values were then standardised to the protein content.

### Protein Quantification:

The protein content was determined using the PierceTM Protein Assay Kit (Thermo Fisher Scientific, Waltham, MA, USA). The instructions provided by the manufacturer were followed. Bovine serum albumin was used as standard. The protocol was based on a publication by Smith et al. (Smith P K, Krohn R I, Hermanson G T et al. (1985) Measurement of protein using bicinchoninic acid. Analytical Biochemistry 150: 76-85).

Nematode (Caenorhabditis elegans)
C. elegans and Bacterial Strains

The C. elegans wild-type strain N2 originates from the Caenorhabditis elegans Genetics Center (University of Minnesota, MN, USA). The nematodes were on nematode growth medium (NGM) agar plates, which were seeded with E. coli OP50 at 20° C. in accordance with standard protocols (Brenner S (1974) The genetics of Caenorhabditis elegans. Genetics 77: 71-94). For all the experiments, synchronous populations were generated by means of a standard bleaching protocol (Theresa Stiernagle (2006) Maintenance of C. elegans. WormBook: 1-11).

### Cultivation and Treatment:

Synchronous larvae were washed twice in M9 buffer, were counted and were adjusted to 10 larvae per 10 μL. The nematodes were pipetted into 96-well plates (Greiner Bio-One, Frickenhausen, Germany). The concentration of OP50 was adjusted to 1 at the optical density OD600 with liquid nematodes. OP50-NGM was added as a standardised nutrition source, with 4.4 times the volume of the larvae having been used with M9 solution. The Li larvae were kept at 20° C. with constant agitation and reached adulthood within 3 days. Effectors were added after reaching young adulthood and 48 hours before the experiment. 100 μM magnesium orotate and 50 μM folic acid, dissolved in M9, were applied as effectors. M9 buffer was applied as a control.

### Heat-Stress Resistance:

As explained above, approximately 10 nematodes were bred per well in a 96-well microplate. After 48 hours of incubation, as described above, effectors were applied. The time until death of the nematodes was determined using a Thermo tolerance test microplate, as described in Fitzenberger et al. (Fitzenberger E, Deusing D J, Marx C et al. (2014) The polyphenol quercetin protects the mev-1 mutant of Caenorhabditis elegans from glucose-induced reduction of survival under heat-stress depending on SIR-2.1, DAF-12, and proteasomal activity. Molecular nutrition & food research 58: 984-994).

The nematodes were washed out of the wells with M9 buffer in 15 ml vials, followed by three additional washing steps. In each well of a black 384-well microplate (Greiner Bio-One, Frickenhausen, Germany), 6.5 μL M9 buffer/Tween® 20 (1% v/v) solution was added. 1 μL M9 buffer was then pipetted into each well with a nematode under a stereomicroscope (Breukhoven Microscope Systems, Capelle aan den Ijssel, The Netherlands) and was mixed with 7.5 μl SYTOXTM green (final concentration 1 M; Life Technologies, Karlsruhe, Germany). To prevent water from evaporating, the plates were sealed with a Rotilabo sealing film (Greiner Bio-One, Frickenhausen, Germany). Thermal shock (37° C.) was induced, and the fluorescence was measured using a ClarioStar plate reader (BMG, Ortenberg, Germany) every 30 minutes over a time period of 17 hours. In order to establish the green fluorescence of SYTOXTM, the stimulation wavelength was set to 485 nm and the emission was detected at 538 nm.

### Example 2

Oxidative Phosphorylation (OxPhosC) of Mitochondria Isolated from the Hippocampus of Transgenic F344-AD Rats after Treatment

After the F344-AD rats are decapitated, the hippocampus is isolated and homogenised at 4° C. After centrifuging steps, the isolated mitochondria are placed into the chamber of a respirometer. By successively adding substrates and inhibitors to the respiratory chain, the influence on the oxygen consumption in the chamber can be measured on a platinum electrode and individual complexes can thus be investigated for their activity (developed from Tina M. Schwarzkopf, Konrad A. Koch, Jochen Klein, Neurodegeneration after transient brain ischemia in aged mice: Beneficial effects of bilobalide, Brain Research, vol. 1529, 5 Sep. 2013, Pages 178-187, with further references).

| Key | Treatment | Dosage | |
|---|---|---|---|
| A, E (control) | No additives | 0 | |
| B | Magnesium orotate | 500 | mg/kg |
| C | Benfotiamine | 300 | mg/kg |
| D, F (cocktail) | Magnesium orotate | 500 | mg/kg |
| | Benfotiamine | 300 | mg/kg |
| | Vitamin B12 | 1 | mg/kg |
| | Folic acid | 10 | mg/kg |
| | Vitamin D3 | 3000 | I.U./kg |

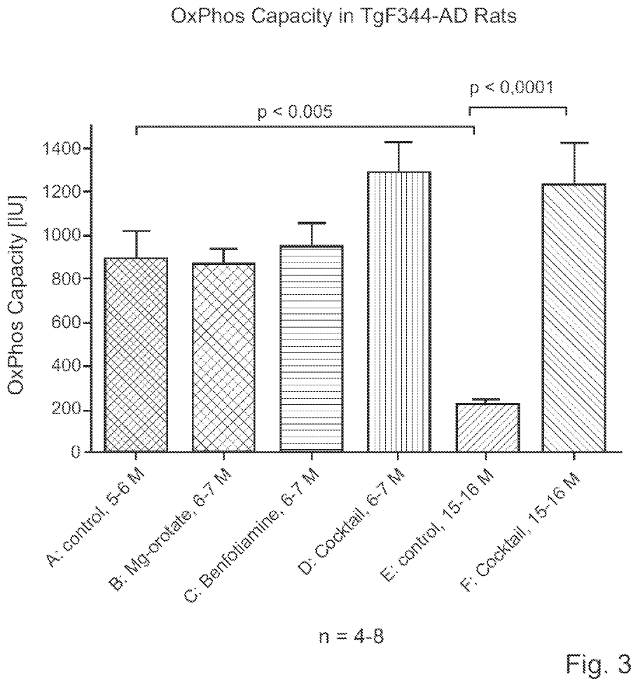

The results are shown in FIG. 3.

FIG. 1 shows the quantity of beta amyloid peptide (AB1-40) in μg/mg protein in SH-SY5Y-APP695 after 24 hours of incubation with the active substances A.) folic acid (folate; 10 μM) and magnesium orotate (200 μM) and combinations thereof and B.) folic acid (folacin; 10 μM) and zinc orotate (200 μM) and combinations thereof. The respective mean values from 6 separate tests (n=6) and the associated standard deviations (±SEM) are shown. The significances (***p<0.001 and ****p<0.0001, respectively) were determined by means of a one-way ANOVA. The dotted line gives the mean of the control (217.4 μg/mg AB).

FIG. 1A shows a synergistic effect for folic acid and magnesium orotate, since:
Δ combination >Δ magnesium orotate+Δ folate.

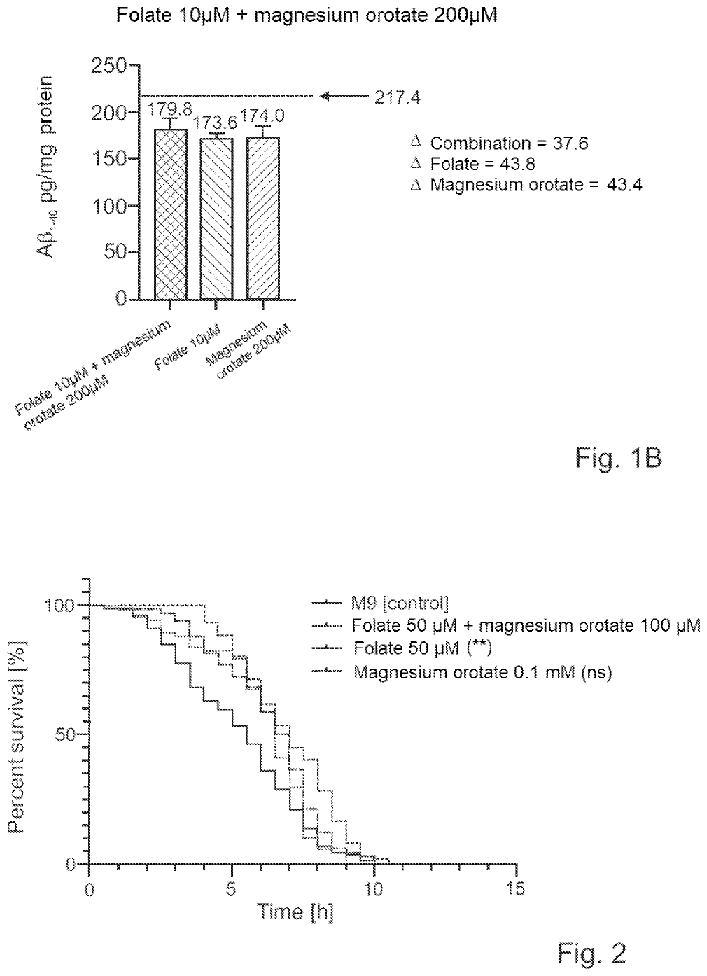

FIG. 2 shows the lifespans of C. elegans in the heat-stress assay after treatment with magnesium orotate and folic acid (“folate”) and combinations thereof in the form of Kaplan-Meier curves. Untreated worms were used as the control (M9 [control]). The respective mean values and the associated standard deviations (±SEM) are shown. The significance (**p<0.01) was calculated by means of a log-rank (Mantel-Cox) test.

FIG. 3 shows the capacity (IU=μmol/min) of the oxidative phosphorylation (OxPhosC) of mitochondria isolated from the hippocampus of transgenic F344-AD rats. The four treatments (B, C, D, F) and the two age groups (6-7 months, 15-16 months) are plotted on the abscissa. Between the 6-7-month-old animals, there are no significant differences in the OxPhosC due to the treatments, even though the treatment with the cocktail (group D) caused an increase in the OxPhosC. The OxPhosC decreases in a statistically significant manner based on age (cf. group A with group E). This effect of age can be influenced or reversed in a statistically significant manner by the treatment with the cocktail (cf. group E with group F).

### REFERENCES

1. Hardy J. A hundred years of Alzheimer's disease research.

### Neuron. 2006; 52(1):3-13.

2. Selkoe D J and Hardy J. The amyloid hypothesis of Alzheimer's disease at 25 years. EMBO Mol Med. 2016; 8(6):595-608.
3. Querfurth H W and LaFerla F M. Alzheimer's disease. N Engl J Med. 2010; 362(4):329-344.
4. Harold D, Abraham R, Hollingworth P, Sims R, Gerrish A, Hamshere M L, Pahwa J S, Moskvina V, Dowzell K, Williams A, Jones N, Thomas C, Stretton A, Morgan A R, Lovestone S, Powell J, et al. Genome-wide association study identifies variants at CLU and PICALM associated with Alzheimer's disease. Nat Genet. 2009; 41(10):1088-1093.
5. Lambert J C, Ibrahim-Verbaas C A, Harold D, Naj A C, Sims R, Bellenguez C, DeStafano A L, Bis J C, Beecham G W, Grenier-Boley B, Russo G, Thorton-Wells T A, Jones N, Smith A V, Chouraki V, Thomas C, et al. Meta-analysis of 74,046 individuals identifies 11 new susceptibility loci for Alzheimer's disease. Nat Genet. 2013; 45(12):1452-1458.
6. Genin E, Hannequin D, Wallon D, Sleegers K, Hiltunen M, Combarros 0, Bullido M J, Engelborghs S, De Deyn P. Berr C, Pasquier F, Dubois B, Tognoni G, Fievet N, Brouwers N, Bettens K, et al. APOE and Alzheimer disease: a major gene with semi-dominant inheritance. Mol Psychiatry. 2011; 16(9):903-907.
7. Naj A C, Jun G, Beecham G W, Wang L S, Vardarajan B N, Buros J, Gallins P J, Buxbaum J D, Jarvik G P, Crane P K, Larson E B, Bird T D, Boeve B F, Graff-Radford N R, De Jager P L, Evans D, et al. Common variants at MS4A4/MS4A6E, CD2AP, CD33 and EPHAl are associated with late-onset Alzheimer's disease. Nat Genet. 2011; 43(5):436-441.
8. Daulatzai M A. Cerebral hypoperfusion and glucose hypometabolism: Key pathophysiological modulators promote neurodegeneration, cognitive impairment, and Alzheimer's disease. J Neurosci Res. 2017; 95(4):943-972.
9. Baglietto-Vargas D, Shi J, Yaeger D M, Ager R and LaFerla F M. Diabetes and Alzheimer's disease crosstalk. Neurosci Biobehav Rev. 2016; 64:272-287.
10. Wild S, Roglic G, Green A, Sicree R and King H. Global prevalence of diabetes: estimates for the year 2000 and projections for 2030. Diabetes Care. 2004; 27(5):1047-1053.
11. Abate G, Marziano M, Rungratanawanich W, Memo M and Uberti D. Nutrition and AGE-ing: Focusing on Alzheimer's Disease. Oxid Med Cell Longev. 2017; 2017:7039816.
12. Qizilbash N, Gregson J, Johnson M E, Pearce N, Douglas I, Wing K, Evans S J and Pocock S J. BMI and risk of dementia in two million people over two decades: a retrospective cohort study. Lancet Diabetes Endocrinol. 2015; 3(6):431-436.
13. Cordain L, Eaton S B, Sebastian A, Mann N, Lindeberg S, Watkins B A, O'Keefe J H and Brand-Miller J. Origins and evolution of the Western diet: health implications for the 21st century. Am J Clin Nutr. 2005; 81(2):341-354.
14. Nishida C, Uauy R, Kumanyika S and Shetty P. The joint WHO/FAO expert consultation on diet, nutrition and the prevention of chronic diseases: process, product and policy implications. Public Health Nutr. 2004; 7(1A):245-250.
15. Janson J, Laedtke T, Parisi J E, O'Brien P, Petersen R C and Butler P C. Increased risk of type 2 diabetes in Alzheimer disease. Diabetes. 2004; 53(2):474-481.
16. Kroner Z. The relationship between Alzheimer's disease and diabetes: Type 3 diabetes? Altern Med Rev. 2009; 14(4):373-379.
17. Clarke J R, Lyra ESNM, Figueiredo C P, Frozza R L, Ledo J H, Beckman D, Katashima C K, Razolli D, Carvalho B M, Frazao R, Silveira M A, Ribeiro F C, Bomfim T R, Neves F S, Klein W L, Medeiros R, et al. Alzheimer-associated Abeta oligomers impact the central nervous system to induce peripheral metabolic deregulation. EMBO Mol Med. 2015; 7(2):190-210.
18. Jais A, Einwallner E, Sharif 0, Gossens K, Lu T T, Soyal S M, Medgyesi D, Neureiter D, Paier-Pourani J, Dalgaard K, Duvigneau J C, Lindroos-Christensen J, Zapf T C, Amann S, Saluzzo S, Jantscher F, et al. Heme oxygenase-1 drives metaflammation and insulin resistance in mouse and man. Cell. 2014; 158(1):25-40.
19. Calay E S and Hotamisligil G S. Turning off the inflammatory, but not the metabolic, flames. Nat Med. 2013; 19(3):265-267.
20. Graham L C, Harder J M, Soto I, de Vries W N, John S W and Howell G R. Chronic consumption of a western diet induces robust glial activation in aging mice and in a mouse model of Alzheimer's disease. Sci Rep. 2016; 6:21568.
21. Rodriguez-Moran M, Guerrero-Romero F2. Oral magnesium supplementation improves the metabolic profile of metabolically obese, normal-weight individuals: a randomized double-blind placebo-controlled trial. Arch Med Res. 2014 July; 45(5):388-93. doi: 10.1016/j.arcmed.2014.05.003. Epub 2014 May 11
22. MISCHOULON DAVID ET AL: “The role of folate in depression and dementia.”, THE JOURNAL OF CLINICAL PSYCHIATRY 2007, vol. 68 Suppl 10, 2007, pages 28-33
23. Wenzhang Wang, Fanpeng Zhao, Xiaopin Ma, George Perry, Xiongwei Zhu, Mitochondria dysfunction in the pathogenesis of Alzheimer's disease: recent advances, Molecular Neurodegeneration (2020) 15:30
24. Simon M. Bell, Katy Barnes, Matteo De Marco, Pamela J. Shaw, Laura Ferraiuolo, Daniel J. Blackburn, Annalena Venneri, Heather Mortiboys, Mitochondrial Dysfunction in Alzheimer's Disease: A Biomarker of the Future? Biomedicines 2021, 9, 63
25. Catrin Herpicha, Kristina Franz, Ursula Müller-Werdan, Mario Ost, Kristina Norman, Age-related fatigue is associated with reduced mitochondrial function in peripheral blood mononuclear cells, Exp. Gerontology 2021, Vol. 144

1. An agent comprising magnesium orotate and folic acid or folates or tautomers and salts thereof.

2. A method for prophylaxis or treatment of dementia selected from the group of Alzheimer's dementia or Alzheimer's disease, dementia in Alzheimer's disease with early onset (type 2), dementia in Alzheimer's disease with late onset (type 1), dementia in Alzheimer's disease, atypical or mixed type, vascular dementia, and Lewy body dementia, said method comprising administering to a patient the agent of claim 1.

3. (canceled)

4. The agent of claim 1, wherein the molar ratio of magnesium orotate to folic acid is 2-25:1.

5. The agent of claim 1, wherein the agent additionally contains at least one or more substances selected from the group containing zinc orotate, benfotiamine, vitamin D and vitamin B.

6. The agent of claim 1, wherein the agent additionally contains at least one or more substances selected from the group containing zinc orotate, vitamin D3, thiamine, vitamin B6, vitamin B12 and benfotiamine.

7. The agent of claim 1, wherein the agent is a drug or a dietary supplement or a balanced diet.

8. A pharmaceutical preparation comprising an agent of claim 1, optionally together with suitable carrier substances.

9. The pharmaceutical preparation of claim 8, together with excipients and additives.

10. A method for prophylaxis or treatment of dementia in a subject, said method comprising administering to the subject the agent of claim 1.

11. A method for prophylaxis or treatment of mitochondrial dysfunction in a subject, said method comprising administering to the subject the agent of claim 1.

12. The method of claim 11, wherein the mitochondrial dysfunction comprises heart failure, non-alcoholic hepatitis, chronic fatigue, chronic fatigue syndrome, or fibromyalgia.

13. The pharmaceutical preparation of claim 8, wherein the preparation is in the form of drops, juice, syrup, tablets, sugar-coated tablets, capsules, sustained-release formulations, infusions, ointments, emulsions, or a powder.
